(19) United States
(12) Patent Application Publication
(10) Pub. No.: US 2021/0401625 A1
(43) Pub. Date: Dec. 30, 2021

(54) Perfect Swab

(71) Applicant: Ronnie Leon Hammers, Phoenix, AZ (US)

(72) Inventor: Ronnie Leon Hammers, Phoenix, AZ (US)

(21) Appl. No.: 16/912,862

(22) Filed: Jun. 26, 2020

(51) Int. Cl. A61F 11/00; A61F 13/38

(52) U.S. Cl. CPC A61F 11/006; A61F 13/38

(57) ABSTRACT

This present patent application discloses a disposable earwax cleaning swab product that removes earwax and debris from the ear easily and safely. The disposable earwax cleaning swab product is comprised of a comfortable plastic handle with a generally spiral, tapered, pyramidal, tapered or conically twisted soft cleaning swab head. The twisted head is made up of foam material for easy and safe removal of earwax from the outer ear canal. Additionally, the earwax swab product has an ear guard which limits a depth of the swab product within the ear of the user. The earwax cleaning swab product eliminates the need for users to purchase costly replacement heads, allowing users to effectively remove a buildup of ear wax and other debris.

### BACKGROUND

The present invention relates generally to the field of earwax cleaners. More specifically, the present invention relates to a disposable one-piece perfect swab product to effectively remove earwax and other debris from a human outer ear folds, lobes and canal. The disposable one-piece integral swab body is comprised of a comfortable plastic handle with a generally spiral, tapered, pyramidal, tapered or conically shaped soft cleaning head made up of a foam material for easy and safe removal of earwax from the outer ear folds, lobes and ear canal. As used herein, the term “perfect swab” refers to the various kinds of disposable earwax swab products known variously as ear swabs, cotton swabs, cotton heads, cotton buds, cotton ear pickers, earwax cleaners and the like. Accordingly, the present specification makes specific reference to the presently described invention, however, it is to be appreciated that aspects of the present invention are also equally amenable to other like applications, devices and methods of manufacture.

By way of background, a cotton swab is composed of a plastic or paper handle with a fibrous head usually cotton, which is placed on either one or both the ends of the handle. A small tube or plastic stem with a very small diameter is produced, and then wrapped around at least one end of the tube or stem with cotton fibers or other woven or non-woven material to form the head of the swab. By lightly inserting one of the cotton head ends into an outer ear canal and rotating or spinning the head end inside the ear canal or outer ear folds and lobes, the user is able to remove some of the cerumen (earwax) and debris collected within the ear canal. However, the cotton heads normally become dirty after one-time use and people may have to use multiple cotton swabs in order to properly remove the cerumen and debris within ear canal.

Cotton swabs are used in virtually every household for a variety of purposes. For example, cotton swabs are used to collect samples of microbiological cultures, and other substances by swiping an area of interest. Cotton swabs are also used to apply a medication, cleaning solution, ointment, and/or other substances to a selected area. Some users apply and/or remove make-up or other cosmetic formulas with cotton swabs. Other users clean small and/or tight spaces, such as a laser head of a CD/DVD player, with cotton swabs. However, the most common use of the cotton swabs is to remove cerumen (earwax, dead skin and secretions) and other ear debris from external ear canals, folds and lobes.

Consumers of cotton swabs for removing earwax, desire effective and safe swab products that they may select from for purchase and subsequent use. In order to fulfill different requirements, the user may have to buy expensive cotton swab products which also require replacement of the cotton head after use to accommodate their needs, which are not only expensive but may consume a large amount of time and cause inconvenience to the user. Further, the user needs to remember that the cotton head is to be bought for using the cotton swab for removing earwax. One cannot use the swab, if the user forgets to buy the cotton swab head. Furthermore, such expensive cotton swabs are unsafe since while using the detachable cotton swabs, the cotton swab heads may remain inside the ear and need to be pulled manually which can be dangerous.

Additionally, conventional cotton swab products oftentimes fail to satisfy the user's wants and needs. More specifically, conventional cotton swabs may require a lot of effort by the user to dislodge the cerumen and debris and could lead to injury if the individual uses too much force in the cleaning activity. People may have to use multiple cotton swabs in order to remove the cerumen. Further, in order to remove the ear wax and debris from the ear canal, the user has to flatten the cerumen before removing it and inadvertently push the cerumen deeper into the ear canal, instead of removing the cerumen. The conventional cotton swabs are unhealthy and may cause ear drum damage, bacterial infection, and other health complications and if not done correctly, the user may actually exacerbate the problem further compacting the ear wax.

An eardrum is easily reached with a cotton swab. Because the eardrum is so delicate, it can be easily ruptured by using even the gentlest pressure when using a swab and pressing to deeply. In severe cases, the cotton swab may damage many sensitive structures behind the ear canal and cause complete deafness, prolonged vertigo with nausea and vomiting, loss of taste function, and even facial paralysis. Accordingly, there is a need for effective and improved cotton swab to remove ear wax and debris which works complimentary to an intricate internal structure of human ear and that is capable of limiting the depth of the cotton swab in the ear to overcome the shortcomings present in the conventional cotton swabs for earwax removal and prevent damage or injury.

Various proposals for an ear cleaning device have been made but none of them appear to be commercially available to the consumer except for devices that are generally is not disposable and sells for an expensive price, for example, about $4.00 or more. The problem with such a permanent, expensive ear cleaning device is that it needs to be cleaned and there is always a danger of ineffective cleaning, leading to potential bacterial contamination and subsequent cutting and a consequent infection. Additionally, the consumers are accustomed to having inexpensive, disposable instruments for which they pay a small price, rather than an expensive implement for cleaning their ears, which, in turn, has to be cleaned.

It is also worth noting that in most cotton swab products that are not disposable, the cotton heads need to be replaced after every use, which is cumbersome, time consuming and expensive for the user. Accordingly, a disposable perfect swab is desired to overcome the disadvantages of the conventional earwax swab products.

Therefore, there exists a long felt need in the art for an earwax cleaning swab product that easily, efficiently and effectively removes earwax and debris from the human ear canals. There is also a long felt need in the art for a earwax cleaning swab that has a head structure that easily conforms to the internal shape of the ear and also does not damage the internal parts such as ear drum of the ear. Additionally, there is a long felt need in the art for a perfect earwax cleaning swab which leads to safety of the user by providing an ear guard element that limits the depth of the cotton swab product can be inserted within the ear of the user. Finally, there is a long felt need in the art for an earwax cleaning swab product that is disposable, and is an integral one body piece structure and inexpensive to use.

The subject matter disclosed and claimed herein, in one embodiment thereof, comprises a comfortable grip plastic handle and a generally spiral tapered, pyramidal, tapered or conically shaped head of foam readily capable of being used by the user to safely remove earwax and debris from the ear canal. More specifically, the improved perfect swab of the present invention allows a user to simply insert the earwax cleaning swab without much inconvenience and with much more safety, thereby allowing the user to effectively remove earwax from the ear canal. Additionally, the improved disposable one body, disposable swab is also aesthetically pleasing and cost effective, as well as being safe for the user by providing an ear guard which limits the depth of the swab product within the ear of the user. The ear guard fits around the shaft of the swab and is generally circular. Other configurates may also be available such as a rod or rods on the “X” and/or “Y” axis of the shaft forming a cross. The invention also offers a more convenient disposable earwax cleaning swab for individuals to use, without replacing the earwax cleaning swab head structure.

In this manner, the perfect swab of the present invention accomplishes all of the forgoing objectives, and provides a relatively safe, easy and cost-effective solution to remove earwax and other debris from the ear canals according to the wants and needs of the user. The improved earwax cleaning swab of the present invention is also user friendly inasmuch as it is disposable, less expensive and does not require the user to remember to purchase the replacement heads. Finally, the improved earwax cleaning swab of the present invention has an ear guard and a soft spiral, tapered, pyramidal, tapered or conically shaped earwax cleaning head that effectively removes earwax and other debris in a safe manner from the ear canals.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an improved disposable earwax cleaning swab product capable of being used to remove earwax, debris, other secretions and the like from ear canals, lobes and folds of a human. More specifically, the disposable earwax cleaning swab of the present invention comprises a comfortable plastic handle with a grip and a generally spiral, tapered, pyramidal, tapered or conically shaped soft head of foam that enables a user to simply and conveniently remove earwax, debris and the like in a safely and inexpensive way. In design, the overall contour of the perfect earwax cleaning swab product resembles dimensions of an external auditory canal and is flexible enough to conform to the canal and any deviations in the ear or canal during insertion. Further, an ear guard component of the earwax cleaning swab of the present invention which lies at a joint section of the plastic handle and the spiral head, prevents the earwax cleaning swab from going deep in the ear canal of the user. The flexible spiral or conically foam shaped head compresses when the earwax cleaning swab product is inserted in the ear and thereby extract earwax and debris as the head expands when the earwax cleaning swab product is taken out.

The subject matter disclosed and claimed herein, in one embodiment thereof, comprises a disposable one piece body integral, disposable cotton swab having an elongate plastic handle to be gripped by a user and having a first end and a second end. An integral circular shaped ear guard member is attached to the second end and an integral generally spiral or cone-shaped soft head portion is attached to the circular shaped ear guard. The elongate plastic handle is flexible and is easy to grip and the cotton swab product and can be easily rotated using the plastic handle. The ear guard member is fixed to the shaft and spaced from the second end of the plastic handle thereby fixing the generally spiral shaped soft cleaning head to the plastic handle. The cone-shaped or spiral cleaning head is not detachable and will always be permanently fixed to the plastic handle thereby preventing the detachment of the cleaning head from the handle while in use. The cone-shaped spiral cleaning head has a distal tip portion and a proximal base portion, that is larger than the distal tip portion which is fixed to the plastic handle. The cone-shaped spiral cleaning head has the largest diameter at the base portion and the smallest diameter at the top portion, having a ratio of 1.5 to 1 to 4 to 1. By inserting the cone-shaped spiral cleaning head into a cavity, such as an ear canal, and gently rotating the flexible handle slowly above cerumen or other debris, the user may easily and safely capture and remove the cerumen and other debris from the cavity, ear folds or lobes. The generally spiral shape of the cleaning head is such that it anatomically fits the external auditory canal and remove the earwax bolus or other debris as a single collection.

The typical human ear canal is about five to six millimeters across in a bony portion near a tympanic membrane and about eight to ten millimeters wide ear a lateral, cartilaginous region. The canal length, about two to three centimeters long, defines a maximum depth of penetration of the generally spiral shaped soft cleaning head. The ear guard membrane is also intended to limit the extent of tip penetration at the narrowing of the canal known as the “bony-cartilaginous junction.”

In yet another embodiment of the present invention, an arrow and/or other instructions may be present on the plastic handle indicating a direction in which a user needs to rotate or twist the earwax cleaning swab of the present invention to remove the earwax and debris from the outer ear canal. Warning information may also be provided to alert the user to other possible concerns. The swab may be provided in any number of colors and the cone shaped head and ear guard may be provided in colors different than the shaft to further differentiate the components of the product.

In a further embodiment of the presently described invention, an ear wax removal tool, is provided and includes an elongated handle having first and second ends and a central portion disposed between the first and second ends. The swab includes a guard positioned along one of the first and second ends and a soft head disposed adjacent the guard. The elongated handle has a gripping surface and the guard having first and second sides with the soft head attached to one of the first and second sides. The soft head having a series of ridges and scraping edges the scraping edges collecting ear wax for the ridges.

In a still further embodiment of the presently described invention, a swab, is described an includes an elongated handle having first and second ends and a central portion between the first and second ends. A guard is disposed on one of the first and second ends, the guard has first and second sides. A soft head element is disposed on one of the first and second heads of the guard and the soft head element has first and second ends with the first end having a smaller diameter than the second end.

In a still further embodiment of the presently described invention, a method of using a swab is provided and includes the steps of initially providing a swab. The swab has a handle with first and second ends and a central portion disposed between the first and second ends. The swab includes a guard positioned along one of the first and second ends and a soft head disposed adjacent the guard, with the soft head having a series of ridges and scraping edges. Next, the swab is inserted into an ear canal and the swab is rotated so that the edges move ear wax or other debris to the ridges and the soft head has a larger proximal end and a smaller distal tip portion such that the larger proximal end removes larger pieces of ear wax and the distal tip portion removes smaller pieces of ear wax. Then the swab and the ear wax and other debris is removed and the swab is disposed of.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one possible embodiment of a swab of the present invention in accordance with the disclosed structure;

FIG. 2 illustrates an exploded view of the generally cone-shaped spiral soft cleaning head of the present invention in accordance with the disclosed structure; and

FIG. 3 illustrates a perspective view of the swab of the present invention effectively removing earwax from a human ear in accordance with the disclosed structure.

### DETAILED DESCRIPTION

The innovation is now described with reference to the drawings, where like reference numerals are used to refer to like elements throughout the specification. In the following description, for purposes of explanation, numerous specific details are set forth in order to provide a general understanding of the invention. It may be evident, however, that the innovation can be practiced without these specific details and modifications are of course possible. In other instances, well-known structures and devices are shown in block diagram form in order to facilitate a description thereof.

As noted above, there exists in the art a long felt need in the art for a disposable, economical and efficient earwax cleaning swab product that easily and effectively removes earwax and other debris from the human ear canals as well as the lobes and folds of the outer ear. There is also a long felt need in the art for an earwax cleaning swab that has a head structure that easily and readily conforms to the internal shape of the ear without significant force and also does not damage the internal parts of the canal such as the ear drum of the ear. Additionally, there is a long felt need in the art for a earwax cleaning swab which leads to the safety of the user by providing an ear guard that limits the depth of the earwax cleaning swab product within the ear of the user through use of a guard element, disposed from the end of the head on the shaft. Finally, there is a long felt need in the art for an earwax cleaning swab product that is disposable, for one-time use only and is an integrally formed, one body piece structure and inexpensive to use.

The innovative product of the present invention features a disposable one piece ear canal cleaning swab product that is comprised of an elongate plastic handle to be gripped by a user and having a first end and a second end. The handle may also be formed from other materials such as paper which may be more environmental preferable or even bamboo which is a renewable resource. The swab further includes an integral, generally circular shaped ear guard member having first and second sides attached to and disposed from the second end of the plastic handle and an integral generally spiral or cone shaped soft head portion attached to the circular shaped ear guard on one side. The ear guard may also be tapered from one side to the other, decreasing in circumference toward the soft head portion. The elongate handle is flexible and is easy to grip and the earwax cleaning swab product can be easily rotated in a convenient and safely manner using the plastic handle as it is inserted into the ear canal or other portions of the ear. The ear guard member is fixed to the second end of the plastic handle thereby fixing the generally spiral shaped soft cleaning head to the plastic handle. The soft cleaning head may be selected from any number of suitable materials, such as a latex rubber foam, other non-allergenic materials, polyurethane foam, woven and non-woven organic and synthetic materials or other materials suitable for use with the present invention. The earwax cleaning swab product of the present invention provides users with an inexpensive, disposable one-piece perfect swab product for which the users pay a small price, rather than an expensive non-disposable implement for cleaning their ears, which, in turn, has to be cleaned.

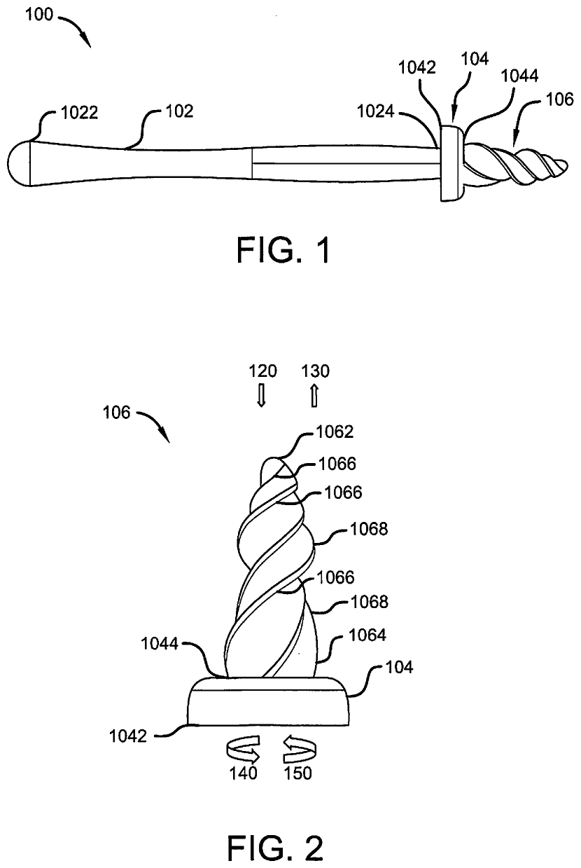

Referring initially to the drawings, FIG. 1 illustrates a disposable one-piece ear wax cleaning swab product 100. The disposable earwax cleaning swab product 100 comprises an elongate plastic, such as polyethylene, PET or other rigid or semi-rigid inexpensive plastic, or paper handle 102 to be gripped by a user having a first end 1022 and a second end 1024 opposite the first end on the handle or shaft. An integrated generally circular or oval shaped ear guard member 104 is attached to and may be spaced from the second end 1024 of the elongate plastic or paper handle 102. The swab further includes an integral generally cone-shaped spiral soft head portion 106 attached to the ear guard 104. A portion of the handle or shaft may also extend partially into the soft head to further secure the head to the shaft or handle. The ear guard 104 is attached to the plastic handle 102 at the end 1042 where a connection is formed between the second end 1024 of the handle 102 and end 1042 of the ear guard 104. The cone-shaped spiral cleaning soft head 106 is attached to an end 1044 of the ear guard 104. The cone-shaped spiral cleaning head 106 has the largest diameter at a base portion which is attached to the side 1044 of the ear guard 104 and the smallest diameter is at a tip portion which is the portion that is inserted into the ear.

In use, a user may insert the soft cleaning head 106 into an ear, lobe or outer fold of the ear, and gently scrape or abrade with the outer ribs and ridges of the cleaning head 106 to allow a general scraping or cleaning action as the handle 102 is twisted or turned. The cone-shaped spiral cleaning head 106 is used to scrape off and remove the ear wax and debris present inside the human ear. The spiral shape of the cleaning head 106 is such that it anatomically fits within an external auditory canal and removes the earwax bolus. Further, a forward or backward axial movement (inserting and retracting) of the cleaning head 106 allows collecting of earwax and debris effectively from the outer ear canal. To provide a good gripping surface in order to be able to rotate the ear cleaning swab device without slipping, it is preferred to provide a grip cover on the handle or shaft body, which may include recesses, ridges either running longitudinally of the shaft or handle, circumferentially or in a pattern on the handle. Further, other gripping patterns may be etched or cut on or into the handle 102 body. In a preferred embodiment, the cleaning head 106 is made of a very soft foam material such as polyurethane and the like which is soft to the internal parts of human ear. The foam material may have an open cell or closed cell to enhance earwax entrapment and collection capabilities and provide a precision and efficient cleaning of the outer ear canal and other ear parts.

In some embodiments, the plastic handle or shaft 102 may have a different shape, such as a prism (triangular, rectangular, pentagonal, etc.) and/or shape, such as curvy, zigzag, wavy, etc. Also, the plastic handle 102 may be hollow or solid and various materials such as plastic, wood, bamboo, wire, rolled paper, and other suitable materials can be used to manufacture the handle 102.

FIG. 2 illustrates an exploded view of the generally cone-shaped spiral soft cleaning head 106 used for cleaning earwax and debris from an ear canal and other ear portions in accordance with the disclosed architecture. As shown in FIG. 2, the cone-shaped spiral cleaning soft head 106 has a distal tip portion 1062 and a proximal base portion 1064 fixed to an end 1044 of the ear guard 104. The soft head may appear undulating, waving or rolling in appearance with the cleaning and scraping edges. The generally spiral soft cleaning head 106 has ridges 1066 and scraping edges 1068 throughout the surface of the cleaning head 106 which helps in efficient cleaning of the earwax from the outer ear canals. The scraping edges 1068 direct the earwax and debris towards the ridges 1066 where the earwax and debris can be retained until the soft cleaning head 106 is removed from the ear. The scraping edges 1068 and ridges 1066 may have different textures, levels of firmness or the like.

A user may bring the cleaning head 106 into contact with surface(s) of an ear and apply linear movement in directions 120 and 130 and/or rotary movement in directions 140 and 150 to engage the scraping edges 1068 with the ear surface(s) and lift or scrape debris from the surface(s) for removal. Thus, the cleaning head 106 is effective at removing wax and other debris using both linear and rotary movement in combination or singularly. The soft cleaning head 106 of the present invention is configured to enable dislodging and removal of cerumen and debris of different sizes, where the proximal base portion 1064 having a larger diameter (containing bigger ridges) is used for removal of larger pieces of cerumen, while the distal tip portion 1062 is used for removal of smaller pieces of cerumen. The user utilizes the cleaning soft head 106 to remove larger, dryer, and stickier pieces of cerumen by placing the cleaning head 106 above the piece of cerumen and pulling it out of the ear canal. Further, the cleaning soft head 106 absorbs moisture from the ear canal, such as after shower or swimming, or remove a smaller piece of cerumen.

As per the embodiment, the cleaning soft head 106 is fixed to the end 1044 of the ear guard structure 104 such that even during the operation by a user, the cleaning soft head 106 does not get detached from the ear guard 104. A portion of the shaft or handle may also extend into the soft head in order to further facilitate fixing of the soft head to the shaft or handle. This ensures the safety of the user such that the cleaning soft head 106 does not remain inside the ear of the user. Any known mechanism such as a glue, adhesive, thermoforming, thermal bonding or as otherwise known in the state of the art may be used to fix the cleaning soft head 106 with the guard structure 104 during the manufacturing of the ear cleaning device 100. The cleaning soft head 106 of the present invention would provide an easy fit for human ear interiors that easily conforms to the internal shape of the ear. In this manner, the cleaning soft head 106 of the present invention proves to be more efficient and safer than a conventional earwax cleaning swab. Additionally, the cleaning soft head 106 does not damage the internal parts such as an ear drum of the ear. Further, the ear guard structure 104 leads to safety of the user by limiting the depth of the cleaning soft head 106 within the ear of the user.

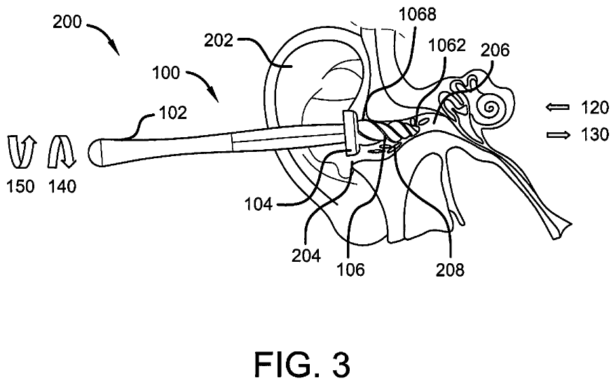

FIG. 3 illustrates an earwax cleaning swab product 100 in use by a user for cleaning earwax or other debris 208 from the outer ear canal 206 or other ear portions. During use, the user grips the shaft or handle 102 and moves the earwax cleaning swab product 100 inwards and outwards in a lateral direction 130 preferably in a direction parallel to the longitudinal direction of the handle or shaft 102. The earwax/cerumen/debris 208 is typically present along the walls of the ear canal 206. During use, once the cleaning soft head 106 reaches the inner of the ear canal 206, the user rotates in a clockwise rotational direction 140 and/or counter-clockwise rotational 150, to effectively scrap and remove the earwax/cerumen/debris 208 using the scraping edges 1068 of the cleaning soft head 106. During use, and to limit the insertion of the earwax cleaning swab product 100 within the ear canal 206, the ear guard 104 touches the Tragus and Antitragus 204 to stop further lateral movement of the earwax cleaning swab product 100 within the ear canal 206. The scraping edges 1068 is configured to rub against and penetrate into cerumen 208, or/and to transfer a portion of cerumen 208 into contact with cleaning head 106, when cleaning head 106 rotates in ear canal 206 around or/and through cerumen 208 in a predetermined rotation direction (clockwise or counterclockwise, in accordance with the helical route of cleaning head 106 around central longitudinal axis, for example).

In one embodiment, the earwax cleaning swab device 100 cleans the earwax and debris inside the ear canal by distally advancing the cleaning head 106 into the ear canal 206 or/and repeating the rotating, thereby scrubbing, scraping and removing cerumen 208 from the ear canal wall with the outer layer and directing the cerumen 208 into contact with the scraping edges 1068 and ridges 1066. The scraping edges 1068 are compressed against a wall of the ear canal 206, thereby increasing total contact area of the cleaning head 106 with the ear canal wall as the soft head is rotated during the cleaning.

The earwax cleaning swab product 100 of the present invention may also be manufactured in a plain white color, or in a variety of different colors, patterns, or decorative prints. The decorative prints could be available in various themes and geometrical shapes such as lines, crosses, rectangles, triangles or sinusoidal lines, sports, logos, designs or a combination of any of these patterns. Further, a customized or personalized print such as a user name and the like could also be available on the plastic handle of the earwax cleaning swab product 100.

In accordance with some embodiments, all of the components of the perfect swab of the present invention are made from environment-friendly materials, such as biodegradable materials. Examples of biodegradable materials that could be used in accordance with embodiments of the present invention include, but are not limited to, paper, biodegradable plastic, wood, bamboo, wire, polyester, rayon fibers, cotton fibers, polyurethane, resins, natural rubbers and foam.

The perfect swab of present invention is not limited to any particular size or any particular color. In some embodiments, an intended user and/or use defines a particular design and size of the perfect swab. For example, the earwax cleaning swab for cleaning a child's ear would generally have a smaller size than an earwax cleaning swab for cleaning an adult's ear. If an earwax cleaning swab is to be used in a surgical procedure, a body and/or end member of different sizes could be used (e.g., longer, shorter, wider, narrower, etc.).

The disposable perfect swab product 100 of present invention may be packaged in perfect swab container packages that may contain 50, 100 or 200 (or any large number as desired by the user) individual cleaning perfect swabs. In addition, travel sized packages may also be available having a reduced number of swabs ranging from 5 to 25 swabs per package. Further, the individual swabs 100 are hygienically stored in the package. The disposable perfect swab product of the present invention promotes more frequent cleaning of the ears and the ear canals due to the inherent features which effectively clean the ears and comfortably and safely remove the excess wax and other debris from inside the ear canal. Further, disposable perfect swab product 100 is a one-time use product and eliminates the need for users to purchase costly replacement cleaning heads as required in the currently available earwax cleaning products while maintaining the internal hygiene of the ear canal.

Although the description above focuses on cleaning of cavities, and ear canals in particular, it should be understood that cleaning an ear canal using the perfect cotton swab is merely an illustrative example and not intended to limit the present invention solely thereto. In accordance with some embodiments, the cotton swab is used to apply substances to a selected area, e.g., ointment, make-up, paint, gel, and the like. As discussed above, in some embodiments, an intended use of the cotton swab defines a particular shape, size, and type of end members used in the cotton swab.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “perfect swab”, “earwax cleaning swab”, “earwax cleaning swab device”, “cleaning swab”, and “swab” are interchangeable and refer to the earwax cleaning swab 100 of the present invention. As used herein “cleaning head” refers to the generally spiral shaped soft earwax cleaning structure. As used herein “cleaning head” and “cleaning foam” are used interchangeably to describe a generally spiral shaped soft cleaning structure to clean earwax from the outer ear canals.

Notwithstanding the forgoing, the earwax cleaning swab 100 of the present invention can be of any suitable size and configuration as is known in the art without affecting the overall concept of the invention, provided that it accomplishes the above stated objectives. One of ordinary skill in the art will appreciate that the size, configuration and material of the earwax cleaning swab as shown in the FIGS. are for illustrative purposes only, and that many other sizes of the earwax cleaning swab 100 are well within the scope of the present disclosure. Although the dimensions of the earwax cleaning swab 100 are important design parameters for user convenience, the earwax cleaning swab 100 may be of any size that ensures optimal performance during use and/or that suits user need and/or preference. It is further contemplated that a single cleaning head of the present invention may have spiral shape with smaller or bigger ridges thereon, and of varying dimensions, thereby allowing the user to choose a particular configuration and size for a specific application. The possibilities are endless.

What is claimed is:

1. A swab, comprising;
an elongated handle having first and second ends and a central portion between the first and second end;
a guard disposed on one of the first and second ends, the guard having first and second sides; and
a soft head element disposed on one of the first and second heads of the guard and the soft head element having first and second ends with the first end having a smaller diameter than the second end.

2. The swab as recited in claim 1, wherein the soft head has a spiral, tapered, pyramidal, or conically twisted shape.

3. The swab as recited claim 1, wherein the soft head is selected from a group of materials including latex rubber foam, non-allergenic materials, polyurethane foam, woven and non-woven organic and synthetic materials or combinations thereof.

4. The swab as recited in claim 1, wherein the soft head first end and second end has a ratio of diameters ranging from of 1 to 1.5 to 1 to 4.

5. The swab as recited in claim 1, wherein the soft cleaning head has a plurality of ridges and scraping edges.

6. The swab as recited in claim 5, wherein the scraping edges direct ear wax and other debris to the ridges for collection and removal.

7. The swab as recited in claim 1, wherein the handle is constructed from materials including paper, biodegradable plastic, wood, bamboo, wire, polyester, rayon fibers, cotton fibers, polyurethane, resins, natural rubbers, foam and combinations thereof.

8. The swab as recited in claim 1, wherein the handle includes a gripping surface.

9. The swab as recited in claim 8, wherein the gripping surface includes recesses, ridges, patterns or combinations thereof.

10. The swab as recited in claim 9, wherein the ridges, patterns or combinations thereof are disposed longitudinally, circumferentially or combinations thereof.

11. The swab as recited in claim 1, wherein the soft head material is selected from open celled foam, closed cell foam or combinations thereof.

12. The swab as recited in claim 1, wherein the swab may be provided in a plain white color, different colors, patterns, or decorative prints.

13. The swab as recited in claim 12, wherein the decorative prints are provided in themes and geometrical shapes such as lines, crosses, rectangles, triangles or sinusoidal lines, sports, logos, designs or a combination thereof.

14. An ear wax removal tool, comprising;
an elongated handle having first and second ends and a central portion disposed between the first and second ends, a guard positioned along one of the first and second ends and a soft head disposed adjacent the guard, and the elongated handle having a gripping surface;
the guard having first and second sides with the soft head attached to one of the first and second sides; and
the soft head having a series of ridges and scraping edges the scraping edges collecting ear wax for the ridges.

15. The ear wax removal tool of claim 14, wherein the soft head may be selected from a group including latex rubber foam, non-allergenic materials, polyurethane foam, woven and non-woven organic or synthetic materials or combinations thereof.

16. The ear wax removal tool of claim 14, wherein the elongated handle is selected from a group including paper, biodegradable plastic, wood, bamboo, wire, polyester, rayon fibers, cotton fibers, polyurethane, resins, natural rubbers and foam or combinations thereof.

17. The ear wax removal tool of claim 14, wherein the tool is single use and disposable.

18. The ear wax removal tool of claim 14, wherein the soft head is composed of open cell or closed cell for earwax or debris collection.

19. The ear wax removal tool of claim 14, wherein the ridges and edges are configured to enable dislodging and removal of ear wax and debris of different sizes, and the soft head has a proximal base portion with a larger diameter ridges for removal of larger pieces of ear wax or debris, and the soft head has a distal tip portion is used for removal of smaller pieces of ear wax or debris.

20. A method of using a swab, comprising the steps of;
providing a swab, the swab having a handle with first and second ends and a central portion disposed between the first and second ends, a guard positioned along one of the first and second ends and a soft head disposed adjacent the guard, the soft head having a series of ridges and scraping edges;
inserting the swab into an ear canal;
rotating the swab so that the edges move ear wax or other debris to the ridges and the soft head has a larger proximal end and a smaller distal tip portion such that the larger proximal end removes larger pieces of ear wax and the distal tip portion removes smaller pieces of ear wax;
removing the swab and the ear wax and other debris; and
disposing of the swab.
